(19) United States
(12) Patent Application Publication
(10) Pub. No.: US 2009/0170815 A1
(43) Pub. Date: Jul. 2, 2009

(54) ALENDRONATE ORAL LIQUID FORMULATIONS

(75) Inventors: Veronica Anne SHANLINE, Dublin, OH (US); Eric Martin SPILLER, Columbus, OH (US)

Correspondence Address: Michael P. Morris;Boehringer Ingelheim USA Corporation, 900 Ridgebury Road, Ridgefield, CT, 06877-0368, US

(73) Assignee: ROXANE LABORATORIES INCORPORATED., Columbus, OH (US)

(21) Appl. No.: 12/342,623

(22) Filed: Dec. 23, 2008

Related U.S. Application Data
(60) Provisional application No. 61/017,306, filed on Dec. 28, 2007.

(51) Int. Cl. A61K 31/66

(52) U.S. Cl. 514/103

(57) ABSTRACT

The invention features an oral pharmaceutical solution comprising a therapeutically effective amount of alendronate or a salt thereof and a pharmaceutically acceptable liquid carrier. The solution is substantially free from degradation products, with the proviso that the solution has no buffer and no complexing agent. The oral solution avoids the difficulties in swallowing tablets of the prior art. Moreover, the oral solution is surprisingly stable without the use of buffering systems and complexing agents of the prior art.

### TECHNICAL FIELD

The present invention is directed to an orally-administrable liquid pharmaceutical composition, or a powder which can be reconstituted into an aqueous solution of a powder, that comprises alendronate, i.e., 4-amino-1-hydroxy-butylidene-1,1-bisphosphonic acid, or salts thereof, in any suitable polymorphic form, including, but not limited to, monosodium trihydrate salt (alendronate sodium), to inhibit bone resorption in human patients.

### BACKGROUND

Normal bones are living tissues undergoing constant resorption and redeposition of calcium, with the net effect of maintenance of a constant mineral balance. The dual process is commonly called “bone turnover”. In normal growing bones, the mineral deposition is in equilibrium with the mineral resorption, whereas in certain pathological conditions, bone resorption exceeds bone deposition, for instance due to malignancy or primary hyperparathyroidism, or in osteoporosis. In other pathological conditions the calcium deposition may take place in undesirable amounts and areas leading to, for example, heterotopic calcification, osteoarthritis, kidney or bladder stones, atherosclerosis, and Paget's disease, which is a combination of an abnormal high bone resorption followed by an abnormal calcium deposition.

Alendronate sodium, 4-amino-1-hydroxybutylidene-1,1-bisphosphonic acid monosodium trihydrate, is an agent for combating bone resorption in bone diseases including osteoporosis and is described as a composition, method of use and synthesis along with other pharmaceutically acceptable salts in U.S. Pat. Nos. 4,922,007 and 5,019,651, incorporated herein by reference. However, there are situations where patients undergoing alendronate sodium therapy for osteoporosis (i.e. rarefaction of bone), for example, experience difficulty in swallowing. Thus, an improved oral formulation to overcome the problem of difficulty in swallowing is desirable.

### TECHNICAL SUMMARY

Orally-administrable pharmaceutical compositions of alendronate, in the form of an aqueous solution, a syrup, or a powder that can be reconstituted into an aqueous solution, offer the advantages of ease of administration and increased compliance for patients who have difficulty swallowing solid oral dosage forms. A powder for reconstitution into an aqueous solution also offers the additional advantage of minimizing storage space in nursing homes, pharmacies, hospitals and warehouses. These formulations have the advantage of permitting dose titration should this be desired.

The present invention provides an oral liquid formulation of alendronate, i.e., 4-amino-1-hydroxy-butylidene-1,1-bisphosphonic acid, or salts thereof or a powder for reconstitution into an aqueous solution. The liquid formulation can be in the form of an aqueous solution or a syrup. In contrast to the known liquid formulations of alendronate, the formulation of the present invention does not contain a buffer system to regulate the pH of the solution. Moreover, the formulation does not contain a complexing agent as taught by the prior art to prevent the formation of insoluble complexes of alendronate. Furthermore, the invention features a method for treating and/or preventing bone loss in a subject who has difficulty in swallowing by administering to the patient a pharmaceutically effective amount of alendronate in a liquid formulation.

The oral liquid pharmaceutical composition of this invention contains a pharmaceutically effective amount of 4-amino-1-hydroxybutylidene-1,1-bisphosphonic acid (alendronate) or salts thereof, in any suitable polymorphic form, including, but not limited to, monosodium trihydrate salt (alendronate sodium), in a liquid pharmaceutically acceptable carrier, e.g. purified water. Also, suitable excipients can include a sweetener, such as sucralose, a flavoring agent such as raspberry blend, and preservatives such as methylparaben and propylparaben. If necessary, the pH of the composition can be adjusted using, for example, sodium hydroxide or hydrochloric acid. In one aspect of the invention, the desired pH range of the formulation will range from about 3.0 to about 6.0 (i.e., in a pH range of 3.3 to 5.7), more specifically above 4.0 and below 5.0. In another aspect of the invention, the pH of the formulation solution will be approximately 4.5.

In an additional aspect of the present invention, the inventors have found that it is possible to formulate stable liquid formulations of alendronate or salts thereof, which are neither highly acidic nor basic, and which do not employ buffer systems contrary to the prior art which requires them. Similarly surprising, the inventors have found it is possible to make liquid formulations of alendronate, or salts thereof, maintained in this pH range substantially free of degradation products without the need for complexing agents that are required by the prior art.

Another aspect of the present invention features a pharmaceutical composition comprising a powder for reconstitution comprising a pharmaceutically effective amount of alendronate or salts thereof in a pharmaceutically acceptable dry excipient capable of dissolving in water.

Turning now to a first embodiment, an oral pharmaceutical solution includes a therapeutically effective amount of alendronate or a salt thereof and a pharmaceutically acceptable liquid carrier. The solution is substantially free from degradation products, with the proviso that the solution has no buffer and no complexing agent.

Referring to specific features of this embodiment, the oral solution is characterized by a pH that is maintained in a range of about 3.0 to about 6.0 (i.e., in a pH range of 3.3 to 5.7), and in particular, from about 4 to about 5. The oral solution can be formed from reconstituting a powder that includes the alendronate and the optional excipient. The alendronate can be in a form of a monosodium trihydrate salt. The oral solution can include at least one excipient, which can include a sweetener, flavoring agent and a preservative. The sweetener can be selected from the group consisting of saccharin, lactose, sucrose, fructose, sucralose, sorbitol, aspartame and combinations thereof. The preservative can be selected from the group consisting of methylparaben, propylparaben, sodium benzoate, sorbic acid, and combinations thereof.

The terms “substantially free from degradation products” mean there are negligible degradation products of the oral solution when stored in a container: for 3 months at 40° C. (±2° C.) and a relative humidity of 75% (±5%), or for 12 months, 24 months or longer at 25° C. (±2° C.) and a relative humidity of 60% (±5%). The oral solution is stable in that it is substantially free from degradation products at a pH maintained in a range of about 3.0 to about 6.0 throughout its shelf life.

Another embodiment features a method of inhibiting bone resorption in a subject in need thereof comprising administering the oral solution to the subject. In another aspect a method of inhibiting bone resorption in a subject in need thereof comprises administering the oral pharmaceutical solution to the subject, wherein the oral solution is administered at a pH in a range of from about 3.0 to about 6.0. In particular, the oral solution can be administered to post menopausal women for the prevention or treatment of osteoporosis. This advantageously is in a form that overcomes the problem of difficulty of swallowing tablets.

Surprisingly, the inventors have found that it is possible to formulate stable, liquid formulations of alendronate, or salts thereof, which are neither highly acidic nor basic, which do not use buffer systems. In the past, buffering systems were considered necessary to maintain the pH of alendronate liquid formulations in the desired range, as disclosed, for example, in U.S. Pat. No. 5,462,932. This patent teaches away from the present invention which features alendronate liquid formulations having a pH that can be maintained in a specified range throughout its shelf life without buffering systems. Examples of buffering systems used in prior art alendronate liquid formulations that are advantageously excluded from the present invention include, but are not limited to, citric acid/sodium citrate, potassium hydrogen tartrate, sodium hydrogen tartrate, potassium hydrogen phthalate, sodium hydrogen phthalate, potassium dihydrogen phosphate and disodium hydrogen phosphate.

Similarly surprising, the inventors have found it is possible to formulate stable liquid formulations of alendronate, or salts thereof, substantially free of degradation products, without the need for complexing agents. Complexing agents were required in prior art alendronate liquid formulations to prevent the precipitation of alendronate through metal complex formation with dissolved metal ions, e.g., Ca, Mg, Fe, Al or Ba, which may leach out of glass containers or rubber stoppers, as disclosed, for example, in U.S. Pat. No. 5,462,932. This patent teaches away from the present invention which features alendronate liquid formulations that are substantially free of degradation products even though they have no complexing agent (e.g., no citrate or EDTA). Further, although buffering systems of prior art liquid alendronate formulations can also function as complexing agents, as discussed above an advantageous feature of the invention is that no buffering systems are used, which excludes such dual function compounds.

### DETAILED DESCRIPTION

The invention is an oral pharmaceutical solution including a therapeutically effective amount of alendronate or a salt thereof and a pharmaceutically acceptable liquid carrier. The solution is substantially free from degradation products, with the proviso that the solution has no buffer and no complexing agent. The oral solution is characterized by a pH that is maintained in a range of 3.3 to 5.7, and in particular, from about 4.0 to about 5.0. The alendronate can be in the form of a monosodium trihydrate salt. The oral solution can include at least one excipient including a sweetener, flavoring agent and a preservative.

There are negligible degradation products of the oral solution when stored in a container: during 3 month accelerated stability testing of the oral solution at 40° C. (±2° C.) and a relative humidity of 75% (±5%), or during stability testing for 12 months, 24 months or longer at 25° C. (±2° C.) and a relative humidity of 60% (±5%). This is discussed in detail in the Examples below.

Pharmaceutically effective amounts in which the alendronate or its salts are administered to humans include an amount sufficient for the treatment and prevention of osteoporosis in postmenopausal woman, an amount sufficient for the treatment to increase bone mass in men with osteoporosis, an amount sufficient for the treatment of glucocorticoid-induced osteoporosis in men and women, an amount sufficient for the treatment of Paget's disease of bone in men and women, and any other amount that is suitable for a conventional pharmaceutical use of alendronate. In particular, the pharmaceutically effective amount is an amount effective to inhibit bone resorption in the foregoing treatments and, more specifically, an amount effective as a specific inhibitor of osteoclast-mediated bone resorption in such treatments. The need to inhibit bone resorption arises locally in cases of bone fracture, non-union, defect, and the like, as well as in cases of systemic bone disease, as in osteoporosis, osteoarthritis, Paget's disease, osteomalacia, multiple myeloma and other forms of cancer, steroid therapy, and age-related loss of bone mass. The oral solution is provided in a 70 mg of alendronate sodium/75 ml single dose bottle. The alendronate can be orally administered as unit dosages according to a schedule having a continuous dosing interval of once weekly dosing, twice weekly dosing, biweekly dosing and thrice monthly dosing until the desired therapeutic effect for the subject is achieved, as disclosed in U.S. Pat. Nos. 5,994,329 and 6,015,801, which are incorporated herein by reference in their entireties.

The term “inhibitor of bone resorption” means treating and preventing bone loss by inhibiting loss of bone from one or more of the mineral phase or organic matrix phase by altering osteoclast formation or activity.

The pharmaceutical formulations of the invention comprise alendronate, or salts thereof, including, but not limited to, 4-amino-1-hydroxybutylidene-1,1-bisphosphonic acid monosodium trihydrate salt (alendronate sodium). Any suitable salts of alendronate can be used in the present invention. The alendronate can be in any suitable polymorphic form and as any hydrate or anhydrous form such as disclosed in the following U.S. Patents all of which are incorporated herein by reference: U.S. Pat. Nos. 7,112,577; 6,963,008; 6,696,601; 6,281,381; 6,008,207; and 5,849,726.

With respect to the excipients that can be utilized in accordance with the invention, carriers, preservatives, sweetener/flavoring agents, solvents and additional ingredients that could adjust the pH of the solution can all be employed.

For liquid formulations, suitable liquid carriers include, but are not limited to, purified water, distilled water, saline solution or the like. Of course, any other conventional liquid carriers can be utilized.

The present invention also can include preservatives. Preservatives which are usable in accordance with the present invention include, but are not limited to, sodium benzoate, sorbic acid, methylparaben and propylparaben and combinations thereof.

The invention also can include sweetener/flavoring agents. Sweetener/flavoring agents which are usable in accordance with the invention include, but are not limited to, saccharin, lactose, sucrose, fructose, sucralose, sorbitol, aspartame and raspberry blend and combinations thereof.

The composition can also include coloring agents/dyes. Examples of suitable coloring agent/dyes include, but are not limited to, FD&C Blue 2 and FD&C Red 33. Other conventional coloring agents/dyes can be employed, if desired.

In addition, the formulation can employ solvents to dissolve the alendronate. Any type of solvent which dissolves alendronate or salts thereof is appropriate. Examples of suitable solvents include, but are not limited to, ethanol, glycerol and propylene glycol.

In addition, the composition optionally includes an additional ingredient to adjust the pH to a range of greater than 4.0 and less than 5.0. Examples of ingredients that are useable to adjust the pH include, but are not limited to, sodium hydroxide and hydrochloric acid. Of course, the sodium hydroxide would be added to raise the pH of the formulation and hydrochloric acid would be added to lower the pH of the formulation.

Another aspect of the present invention features a pharmaceutical composition comprising a powder for reconstitution containing a pharmaceutically effective amount of alendronate sodium in an optional pharmaceutically acceptable dry excipient capable of dissolving in water. The reconstitutable powder formulations of the invention have the advantage of permitting dose titration should this be desired. The powder blend of the invention is placed into multi-dose or unit-dose containers which can be stored for future reconstitution. The powder can be dissolved in distilled water, or any pharmaceutically acceptable solvent that is suitable for a pharmaceutical formulation. The reconstitutable powder formulations are advantageously prepared together with dry inert carriers for the powder form including, but not limited to, sugars, including sucrose and lactose, starch, derivatives of the foregoing (including sucralose), cellulose and derivatives, gums, fatty acids and their salts and the like.

These and other aspects of the invention will be apparent to those of ordinary skill in the art from the following non-limiting examples, which are merely illustrative of embodiments of the present invention, and are not to be construed as limiting the invention.

### EXAMPLE 1

The alendronate sodium oral solution composition can be manufactured as follows, all amounts that are added being shown in Table 1 below. Water is heated in a process tank to approximately 85° C. The methylparaben and propylparaben are added to the tank, while mixing, and mixed until dissolved. The solution is cooled to room temperature (20-30° C.). The sucralose is added to the tank, while mixing, and mixed until dissolved. The alendronate sodium is added to the tank, while mixing, and is mixed until dissolved. The raspberry blend is added to the tank, while mixing, and is mixed until uniform. Purified water is added to the tank until the final batch weight is reached.

In addition, the following additional steps can be conducted either prior to or after the addition of purified water. The pH of the solution is determined. If the pH is higher than the desired range, 0.1 N hydrochloric acid is added to the alendronate sodium oral solution, while mixing, until a desired pH is achieved. If the pH is lower than the desired range, 0.1N sodium hydroxide is added to the alendronate sodium oral solution, while mixing, until a desired pH is achieved.

TABLE 1
Alendronate Sodium Oral Solution, 70 mg/75 ml

| Ingredients | (mg/75 mL) | (% w/v) |
|---|---|---|
| Alendronate Sodium, USP | 91.35 mg | 0.122% |
| Sucralose, NF | 30.0 mg | 0.04% |
| Raspberry Blend | 150.0 mg | 0.20% |
| Methylparaben, NF | 135.0 mg | 0.18% |
| (Methyl Parahydroxybenzoate) | | |
| Propylparaben, NF | 15.0 mg | 0.02% |
| (Propyl Parahydroxybenzoate) | | |
| Sodium Hydroxide Pellets | ** | ** |
| Hydrochloric Acid | ** | ** |
| Water, Purified, USP | QS | QS |

** optionally added, if needed, during processing to adjust pH

### EXAMPLE 2

The alendronate sodium oral solution composition can be manufactured as follows, all amounts that are added being shown in Table 2 below. Water is heated in a process tank to approximately 70° C. The methylparaben and propylparaben are added to the tank, while mixing, and mixed until dissolved. The sucralose is added to the tank, while mixing, and mixed until dissolved. The alendronate sodium is added to the tank, while mixing, and is mixed until dissolved. The solution is cooled to room temperature (20-30° C.). The raspberry blend is added to the tank, while mixing, and is mixed until uniform. Purified water is added to the tank until the final batch weight is reached.

TABLE 2
Alendronate Sodium Oral Solution, 70 mg/75 mL

| mg. per | | |
|---|---|---|
| 75 mL | % w/w | Batch Formula Ingredients |
| 91.35 | 0.122 | Alendronate Sodium, USP |
| | 0.040 | Sucralose |
| | 1.00 | Raspberry Blend |
| | 0.180 | Methylparaben |
| | 0.020 | Propylparaben |
| | 98.638 | Purified Water, USP |

### EXAMPLE 3

The process described in Example 1 is carried out using the compounds in the amounts shown in Table 3 below.

TABLE 3
Alendronate Sodium Oral Solution, 70 mg/75 mL

| mg. per | | |
|---|---|---|
| 75 mL | % w/w | Batch Formula Ingredients |
| 91.35 | 0.122 | Alendronate Sodium, USP |
| | 0.040 | Sucralose |
| | 0.20 | Raspberry Blend |
| | 0.180 | Methylparaben |
| | 0.020 | Propylparaben |
| | 99.438 | Purified Water, USP |

### EXAMPLE 4

The process described in Example 1 is carried out using the compounds in the amounts shown in Table 4 below.

TABLE 4
Alendronate Sodium Oral Solution, 70 mg/75 mL

| mg. per | | |
|---|---|---|
| 75 mL | % w/w | Batch Formula Ingredients |
| 91.35 | 0.122 | Alendronate Sodium, USP |
| | 0.040 | Sucralose |
| | 0.20 | Raspberry Blend |
| | 0.108 | Methylparaben |
| | 0.012 | Propylparaben |
| | 99.518 | Purified Water, USP |

### EXAMPLE 5

The process described in Example 1 is carried out using the compounds in the amounts shown in Table 5 below.

TABLE 5
Alendronate Sodium Oral Solution, 70 mg/75 mL

| mg. per | | |
|---|---|---|
| 75 mL | % w/w | Batch Formula Ingredients |
| 91.35 | 0.122 | Alendronate Sodium, USP |
| | 0.040 | Sucralose |
| | 0.20 | Raspberry Blend |
| | 0.144 | Methylparaben |
| | 0.016 | Propylparaben |
| | 99.478 | Purified Water, USP |

### EXAMPLE 6

Alendronate sodium oral solution described herein, 70 mg/75 mL, is added to a process tank. The pH of the solution is determined. While mixing, 0.1N hydrochloric acid is added to the alendronate sodium oral solution until a desired lower pH is achieved. The amount of alendronate sodium oral solution and hydrochloric acid that were added are shown in Table 6 below.

TABLE 6
Alendronate Sodium Oral Solution, 70 mg/75 mL-To lower pH

| Amount | | Batch Formula Ingredients |
|---|---|---|
| 3000 | g | Alendronate Sodium Oral Solution, 70 mg/75 mL |
| 3.2 | mL | 0.1N Hydrochloric Acid |

### EXAMPLE 7

Alendronate sodium oral solution described herein, 70 mg/75 mL, is added to a process tank. The pH of the solution is determined. While mixing, 0.1 N sodium hydroxide is added to the alendronate sodium oral solution until a desired higher pH is achieved. The amount of alendronate sodium oral solution and sodium hydroxide that were added are shown in Table 7 below.

TABLE 7
Alendronate Sodium Oral Solution, 70 mg/75 mL-To raise pH

| Amount | | Batch Formula Ingredients |
|---|---|---|
| 3000 | g | Alendronate Sodium Oral Solution, 70 mg/75 mL |
| 12.5 | mL | 0.1N Sodium Hydroxide |

### EXAMPLE 8

### Accelerated Stability Test:

Alendronate sodium oral solution, 70 mg/75 ml, having the formulation of Example 1, was subjected to accelerated stability testing at 40° C. (±2° C.) at a relative humidity of 75% (±5%) in a container stored on its side. The results were taken initially and then at 1 month, 2 months and 3 months as shown below. The container was an HDPE bottle. The closure was a polypropylene inner cap with glued liner.

TABLE 8
Alendronate Sodium Oral Solution, 70 mg/75 ml

| Test | Initial | 1 month | 2 months | 3 months |
|---|---|---|---|---|
| Description | Conforms | Conforms | Conforms | Conforms |
| Assay of | 99.9 | 100.4 | 99.1 | 99.8 |
| Alendronate | | | | |
| (% LA) | | | | |
| Degradation | | | | |
| Products | | | | |
| Individual | <0.1 | 0.1 | 0.1 | <0.1 |
| Impurities | | | | |
| RRT @ 0.64 | | | | |
| (% w/w) | | | | |
| Single Largest | 0.1 | <0.1 | <0.1 | <0.1 |
| Unspecified | | | | |
| Degradant | | | | |
| (% w/w) | | | | |
| Total | 0.1 | 0.1 | 0.1 | BQL |
| Degradants | | | | |
| (% w/w) | | | | |
| Specified | 1 | <1 | <1 | 1 |
| Leachable | | | | |
| RRT @ 1.39 | | | | |
| (ppm) | | | | |
| Preservative | | | | |
| Testing | | | | |
| Methylparaben | 99.5 | 100.0 | 99.2 | 98.7 |
| Propylparaben | 99.4 | 98.1 | 97.1 | 95.3 |
| pH | 4.5 | 4.6 | 4.4 | 4.5 |

ND = Not detected
BQL = Below quantitation limit (<0.1%)
RRT = relative retention time on an HPLC column
LA = labeled amount

As can be seen from Table 8, there is little change in pH from the initial pH of 4.5 over the 3 months duration of the test. Moreover, negligible degradation products were formed during that time. These results were achieved even though no buffer or complexing agent were used in the oral formulation.

### EXAMPLE 9

### Accelerated Stability Test

Alendronate sodium oral solution, 70 mg/75 ml, having the formulation of Example 6, was subjected to accelerated stability testing at 40° C. (±2° C.) at a relative humidity of 75% (±5%) in a container stored on its side. The results were taken initially and then at 1 month, 2 months and 3 months as shown below. The same container and closure described in Example 8 were used.

TABLE 9
Alendronate Sodium Oral Solution, 70 mg/75 ml

| Test | Initial | 1 month | 2 months | 3 months |
|---|---|---|---|---|
| Description | Conforms | Conforms | Conforms | Conforms |
| Assay of | 98.9 | 98.9 | 99.5 | 98.4 |
| Alendronate | | | | |
| (% LA) | | | | |
| Degradation | | | | |
| Products | | | | |
| Individual | 0.1431 | ND | ND | 0.1038 |
| Impurities | | | | |
| RRT @ 0.64 | | | | |
| (% w/w) | | | | |
| Single Largest | BQL | BQL | BQL | BQL |
| Unspecified | | | | |
| Degradant | | | | |
| (% w/w) | | | | |
| Total | 0.1431 | BQL | BQL | 0.1038 |
| Degradants | | | | |
| (% w/w) | | | | |
| Specified | 2 | 0 | 0 | 0 |
| Leachable | | | | |
| RRT @ 1.39 | | | | |
| (ppm) | | | | |
| Preservative | | | | |
| Testing | | | | |
| Methylparaben | 99.3 | 99.3 | 98.6 | 97.8 |
| Propylparaben | 99.5 | 97.2 | 96.3 | 94.4 |
| pH | 3.3 | 3.2 | 3.2 | 3.3 |

ND = Not detected
BQL = Below quantitation limit (<0.1%)
RRT = relative retention time on an HPLC column
LA = labeled amount

As can be seen from Table 9, there is little change in pH from the initial pH of 3.3 over the 3 months duration of the test. Moreover, negligible degradation products were formed during that time. These results were achieved even though no buffer or complexing agent were used in the oral formulation.

### EXAMPLE 10

### Accelerated Stability Test:

Alendronate sodium oral solution, 70 mg/75 ml, having the formulation of Example 7, was subjected to accelerated stability testing at 40° C. (±2° C.) at a relative humidity of 75% (±5%) in a container stored on its side. The results were taken initially and then at 1 month, 2 months and 3 months as shown below. The same container and closure described in Example 8 were used.

TABLE 10
Alendronate Sodium Oral Solution, 70 mg/75 ml

| Test | Initial | 1 month | 2 months | 3 months |
|---|---|---|---|---|
| Description | Conforms | Conforms | Conforms | Conforms |
| Assay of | 99.6 | 100.1 | 98.7 | 100.5 |
| Alendronate | | | | |
| (% LA) | | | | |
| Degradation | | | | |
| Products | | | | |
| Individual | ND | ND | 0.132 | BQL |
| Impurities | | | | |
| RRT @ 0.64 | | | | |
| (% w/w) | | | | |
| Single Largest | BQL | BQL | BQL | BQL |
| Unspecified | | | | |
| Degradant | | | | |
| (% w/w) | | | | |
| Total | BQL | BQL | 0.132 | BQL |
| Degradants | | | | |
| (% w/w) | | | | |
| Specified | 3 | 0 | 0 | 0 |
| Leachable | | | | |
| RRT @ 1.39 | | | | |
| (ppm) | | | | |
| Preservative | | | | |
| Testing | | | | |
| Methylparaben | 99.5 | 98.6 | 97.8 | 97.9 |
| Propylparaben | 100.0 | 96.6 | 95.1 | 93.8 |
| pH | 5.7 | 5.6 | 5.6 | 5.6 |

ND = Not detected
BQL = Below quantitation limit (<0.1%)
RRT = relative retention time on an HPLC column
LA = labeled amount

As can be seen from Table 10, there is little change in pH from the initial pH of 5.7 over the 3 months duration of the test. Moreover, negligible degradation products were formed during that time. These results were achieved even though no buffer or complexing agent were used in the oral formulation.

### EXAMPLE 11

### Room Temperature Stability Test:

Alendronate sodium oral solution, 70 mg/75 ml, having the formulation of Example 1, was subjected to stability testing at 25° C. (±2° C.) at a relative humidity of 60% (±5%) in a container stored on its side. The results were taken initially and then at 3 months, 6 months, 9 months and 12 months as shown below. The same container and closure described in Example 8 were used.

TABLE 11
Alendronate Sodium Oral Solution, 70 mg/75 ml

| Test | Initial | 3 months | 6 months | 9 months | 12 months |
|---|---|---|---|---|---|
| Description | Con- | Conforms | Conforms | Conforms | Conforms |
| | forms | | | | |
| Assay of | 99.9 | 100.1 | 99.6 | 100.8 | 99.5 |
| Alendronate | | | | | |
| (% LA) | | | | | |
| Degradation | | | | | |
| Products | | | | | |
| Individual | <0.1 | <0.1 | <0.1 | <0.1 | <0.1 |
| Impurities | | | | | |
| RRT @ 0.64 | | | | | |
| (% w/w) | | | | | |
| Single Largest | 0.1 | <0.1 | <0.1 | 0.1 | <0.1 |
| Unspecified | | | | | |
| Degradant | | | | | |
| (% w/w) | | | | | |
| Total | 0.1 | BQL | BQL | 0.1 | BQL |
| Degradants | | | | | |
| (% w/w) | | | | | |
| Specified | 1 | 1 | 1 | 3 | <1 |
| Leachable | | | | | |
| RRT @ 1.39 | | | | | |
| (ppm) | | | | | |
| Preservative | | | | | |
| Testing | | | | | |
| Methylparaben | 99.5 | 98.8 | 98.9 | 98.9 | 99.5 |
| Propylparaben | 99.4 | 97.3 | 96.9 | 96.6 | 96.1 |
| pH | 4.5 | 4.8 | 4.5 | 4.5 | 4.4 |

ND = Not detected
BQL = Below quantitation limit (<0.1%)
RRT = relative retention time on an HPLC column
LA = labeled amount

As can be seen from Table 11, there is little change in pH from the initial pH of 4.5 over the 12 month duration of the test. Moreover, negligible degradation products were formed during that time. These results were achieved even though no buffer or complexing agent were used in the oral formulation.

The invention has been described hereinabove using specific examples. However, it will be understood by those skilled in the art that various alternatives may be used and equivalents may be substituted for elements or steps described herein, without deviating from the scope of the invention. Modifications may be necessary to adapt the invention to a particular situation or to particular needs without departing from the scope of the invention.

What is claimed is:

1. An oral pharmaceutical solution comprising a therapeutically effective amount of alendronate or a salt thereof and a pharmaceutically acceptable liquid carrier, wherein said solution is substantially free from degradation products, with the proviso that said solution has no buffer and no complexing agent.

2. The oral solution of claim 1 wherein said solution is characterized by a pH that is maintained in a range of 3.0 to 6.0.

3. The oral solution of claim 1 further comprising at least one pharmaceutically acceptable excipient.

4. The oral solution of claim 3 formed from reconstituting a powder that includes said alendronate and said excipient.

5. The oral solution of claim 1 wherein said alendronate is in a form of a monosodium trihydrate salt.

6. The oral solution of claim 3 wherein said excipient includes a sweetener, flavoring agent and a preservative.

7. The oral solution of claim 6 wherein said sweetener is selected from the group consisting of saccharin, lactose, sucrose, fructose, sucralose, sorbitol, aspartame and combinations thereof.

8. The oral solution of claim 6 wherein said preservative is selected from the group consisting of methylparaben, propylparaben, sodium benzoate, sorbic acid, and combinations thereof.

9. The oral solution of claim 1 comprising negligible degradation products when stored in a container for 3 months at a temperature in a range of 38-42° C. and at a relative humidity in a range of 70-80%.

10. A method of inhibiting bone resorption in a subject in need thereof comprising administering said oral solution of claim 1 to said subject.

11. A method of inhibiting bone resorption in a subject in need thereof comprising administering an oral pharmaceutical solution to said subject, said oral solution comprising a therapeutically effective amount of alendronate or a salt thereof and a pharmaceutically acceptable liquid carrier, wherein said solution is substantially free from degradation products, with the proviso that said solution has no buffer and no complexing agent, wherein said oral solution is administered at a pH in a range of from about 3.0 to about 6.0.

12. The method of claim 11 comprising negligible degradation products when stored in a container for 3 months at a temperature in a range of 38-42° C. and at a relative humidity in a range of 70-80%.
